(19) United States
(12) Patent Application Publication
(10) Pub. No.: US 2008/0119587 A1
(43) Pub. Date: May 22, 2008

(54) METHOD OF FORMING RESIN-BASED ARTICLE AND ARTICLE FORMED THEREFROM

(76) Inventor: Johnny Snyder, China Springs, TX (US)

Correspondence Address: NAMAN, HOWELL, SMITH & LEE, P.O. BOX 1470, WACO, TX, 76703, US

(21) Appl. No.: 11/561,278

(22) Filed: Nov. 17, 2006

(51) Int. Cl. A61F 2/52; A61F 2/50

(52) U.S. Cl. 523/113; 427/2.1

(57) ABSTRACT

The present invention is directed toward an improved method of forming resin based articles and the articles formed therefrom. In particular, the present invention provides an efficient, low cost method of forming human body prosthetics, such as external breast prostheses as well as various pads and cushions for healthcare related items. The method uses a resin-based elastomer diluted into a form which can be sprayed onto an open mold cavity. This first layer of elastomer is cured. Then a resin-based filler material is applied to the first layer of elastomer and cured. Finally, a second layer of elastomer is applied to the filler material and cured. The resulting article being a gelatinous or foam resin-based article sheathed in an elastomeric skin.

### BACKGROUND OF THE INVENTION

1. Field of the Invention

The present invention is directed toward an improved method of forming resin-based articles and the articles formed therefrom. In particular, the present invention provides an efficient, low cost method of forming human body prosthetics, such as external breast prostheses as well as various pads and cushions for healthcare related items.

2. Background Information

Many human prosthetics are made of a synthetic silicone resin cured to a gelatinous state with the outer surface of the prosthesis molded to simulate the shape of the particular replacement body part. For instance, external breast prostheses are formed by a process of adding uncured silicone gel to the cavity of a mold modeled in the shape of a human breast and appropriately curing to obtain the look, shape and feel of an actual human breast.

As disclosed in U.S. Pat. No. 5,035,758 issued to Degler, a distinction is made between film-free breast prostheses and prostheses encapsulated or sheathed in film. Film-free breast prostheses have the disadvantage that silicone oil often seeps from the prosthesis because the silicone resin composition often does not crosslink completely, and it is this uncrosslinked silicone that leaks out. This leakage leaves an undesirable sticky or tacky residue on the surface of the prosthesis.

In order to overcome this disadvantage, breast prostheses are typically sheathed in thermoplastic films, such as polyurethane films. In general, such breast prostheses are produced by placing the uncrosslinked silicone resin composition together with a crosslinking agent and a catalyst between two flat films that form an envelope for the prosthesis. The films are welded together along this edge except for a small opening reserved for filling the envelope. The films are then fixed at the edge of a cavity in the area of the welded edge in a die that corresponds to the shape of a human breast. Silicone resin composition is added until the films are pressed against the walls of the die cavity; the film edges are then welded together in the area of the filling opening, and the silicone resin composition is cured to form a gelatinous mass.

However, welding the film edges together especially in the area of the filling opening, poses problems when residues of the injected silicone resin composition are between the films. These residues prevent satisfactory welding of the film edges so the weld seam easily tears open and the silicone resin composition easily escapes during the curing process as well as after the curing process, even when only a slight pressure is applied to the prosthesis. Furthermore the dies must be heated to a relatively high temperature in the welding and crosslinking operation and must be cooled between each step, which is very time consuming and expensive.

As disclosed in U.S. Pat. No. 5,370,688 issued to Schulz et al on Dec. 6, 1994, an example of the typical, time-consuming method of forming an external breast prosthesis is as follows. First, a flexible film of thermoplastic material is heated and placed on a male (convex) vacuum forming tool or mold in order to form the outer skin of the prosthesis. The most commonly used thermoplastic materials are polyurethane based. The male vacuum forming mold is shaped to simulate the natural shape of the female breast and may be of various shapes and sizes in order to produce various sized prostheses. The heated film is then placed over the vacuum forming mold to produce the outer skin of the prosthesis.

The inner skin of an external breast prosthesis is also typically formed by heating and placing a flexible thermoplastic film over a male vacuum forming mold. The inner skin and the outer skin are then sealed together along their peripheries by a method as known in the art, such as a high frequency electronic sealing method of welding the thermoplastic films.

Next, the empty capsule is filled with a gel-forming liquid composition through an opening to form a filled capsule. The opening may be formed by puncturing a small hole in the wall of the capsule or by leaving a small segment of the respective peripheries of the inner skin and outer skin unsealed. Any trapped air in the filled capsule is removed before sealing the opening by the use of a vacuum chamber or other mechanical means. After the trapped air is substantially removed the opening is sealed.

The filled capsule is then placed into a heat chamber where the gel-forming composition is cured to form a gel which comprises the body of the prosthesis. After the gel-forming composition has been cured, the excess film is trimmed from the peripheral edges, producing the completed prosthesis.

In view of the limitations associated with the prior art, a substantial need exists for a method of forming a resin-based article that is less expensive and less time consuming than current methods while retaining the benefits of a prosthesis sheathed in film. Applicant's invention, through a novel combination steps and materials, provides such a method.

### SUMMARY OF THE INVENTION

The general purpose of the present invention, which will be described subsequently in greater detail, is to provide a method for forming resin-based articles, such as human prosthetics or medical cushions, which contains many of the advantages of the prior art along with significant novel features that result in a method that is not anticipated, rendered obvious, suggested, or even implied by any of known method, either alone or in combination.

In view of the foregoing, it is an object of the present invention to provide a method of forming a resin-based article that is less expensive than existing methods.

It is another object of the present invention to provide a method of forming a resin based article that is less time consuming than existing methods.

It is another object of the present invention to provide a method of forming a resin-based article that reduces cycle time of forming said article.

It is another object of the present invention to provide a method of forming a resin-based article that retains the benefits of a resin-based article sheathed in film.

In satisfaction of these and other related objectives, the present invention provides a method for forming a resin-based article, such as human prostheses or cushions and pads for medical products. As will be discussed in the specification to follow, practice of the present invention provides a method, which eliminates the steps of forming and welding thermoplastic sheathing, while retaining the benefits associated with resin-based articles sheathed in film.

The preferred embodiment of the present invention provides a resin-based elastomer, which can be sprayed onto the cavity of the mold itself, thus eliminating thermoplastic sheathing in its entirety. The process of the present invention is as follows. First the appropriate (concave) mold is selected for the application. For example, if the article being manufactured is an external breast prosthesis, the mold should be shaped in the form of a human breast. Next, the appropriate resin-based elastomer is selected for the application. For instance, a spray silicone-based elastomer would be selected for a silicone gel filled external breast prosthesis. The resin-based elastomer is appropriately diluted and prepared into a sprayable form. Next, the elastomer is sprayed onto the mold over a time period to provide adequate coverage of the mold, i.e. for a thicker surface film, the elastomer should be sprayed longer than for a thinner surface film. Once the mold is adequately coated, the mold is placed into a heat chamber at an appropriate temperature for an adequate time period to cure the elastomer into an external ‘skin’ or sheathing for the article. The mold is then removed, and a resin-based gel or foam is added to the mold. The gel or foam must be allowed to completely ‘air out’, ensuring undesired air bubbles are removed prior to curing. The mold is then placed back into the heat chamber for a brief period in order to allow the gel or foam to cure to a gelatinous state. Once, the gel or foam has cured, the mold is removed from the heat chamber, and an additional coating of elastomer is sprayed onto the rear of the article. The mold is then placed back into the heat chamber for a final cure of the second layer of elastomer ‘skin’.

In summary, then, an embodiment of the present invention provides a highly cost-effective and time-saving method of forming resin-based articles, such as human prosthetics or medical product cushions or pads, while retaining the performance of a resin-based article formed with thermoplastic sheathing.

### BRIEF DESCRIPTION OF THE DRAWINGS

Applicant's invention may be further understood from a description of the accompanying drawing.

FIG. 1 is a cross-sectional view of the article of the present invention.

### DETAILED DESCRIPTION OF THE PREFERRED EMBODIMENT

At the outset, it should be understood that the present invention encompasses a method for forming any number of resin-based articles. Such articles may be composed of various materials, examples of which shall be included in the specification to follow. In addition, the resin-based articles which may be formed by said process are envisioned to fall primarily in the medical industry, examples of which shall be included in the specification to follow as well. However, in each case the embodiments and materials expressed are intended as examples only, and should not be viewed in a limited sense.

An embodiment of the present invention that may be most easily understood is with reference to the formation of a gelatinous, human prosthetic, such as an external breast prosthesis. The first step in the formation of an external breast prosthesis begins with the formation or selection of a suitable concave, open faced mold fashioned in a shape that realistically mimics a female breast. Such a mold may be formed in various shapes and sizes as desirable for the final product. Additionally, such molds may be fashioned from epoxy, aluminum, styrene, or any number of other materials, as known in the art.

Next, a suitable resin-based compound is selected to create the skin or covering of the prosthesis itself. The compound selected must be of such a nature and material characteristic as to create a sufficient bond with the filler material selected to form the body of the article itself. For instance, if the material selected to form the body of the external breast prosthesis is a silicone gel, then the elastomer selected to form the skin should also be silicone based. However, if the article being formed is a medical cushion, a foam, such as a polyurethane foam may compose the body of the cushion. In such a case, the elastomer selected should be a polyurethane-based elastomer. Additionally, it is desirable that the materials selected have a similar cure temperature as well to add efficiency to the process. Whatever the composition, this compound must be prepared into a dilute liquid form such that it can be easily painted or sprayed. In the preferred embodiment, the compound is a clear, pourable, two-component silicone rubber compound designed for making flexible parts, having a low durometer and excellent flexibility. In the preferred embodiment, the elastomer is mixed with a crosslinker; then, a solvent such as toluene, xylene, or mineral spirits is added to the elastomer mix in an amount necessary for proper dilution into a paintable or sprayable form.

The next step in the process of forming the prosthesis is to apply the diluted elastomer onto the concave surface of the mold itself in a uniform manner for the appropriate duration to provide proper coverage of the mold. Application of the elastomer to the cavity of the mold may be accomplished by painting or spraying the diluted elastomer directly onto the surface of the mold. This process may be accomplished remotely, robotically, or by hand (observing appropriate safety procedures as dictated by the composition of the elastomer), and the duration of the spray necessary directly corresponds with the desired thickness of the outer ‘skin’ of the prosthesis. For instance, a longer spray duration will obviously result in a thicker layer of elastomer than a shorter spray duration. Additionally, although the preferred embodiment presents a uniform spray of the mold, embodiments are also envisioned wherein, the duration of the spray is more concentrated in specific areas of the mold in order to provide a thicker ‘skin’ in certain areas of the prosthesis than in other areas, a trait which is difficult, if not impossible, using methods derived from the prior art.

At this point, the elastomer must be appropriately cured in order to properly form the ‘skin’ of the prosthesis. For example, most silicone rubber elastomers should be cured with heat; therefore, the elastomer covered mold would be placed into a heat chamber pre-heated to the appropriate temperature and allowed to cure for an appropriate time for the elastomer to cure to the desired state. An example of a proper curing temperature and time for a silicone elastomer is 85° C. for five minutes.

After the elastomer has cured, the mold is removed from the oven, and the resin-based filler material is added. As previously mentioned, this material should not only be selected for its structural characteristics to give the desired look and feel to the article, but it must also be of a composition that will bond with the elastomer selected as well. In the present example of an external breast prosthesis, the filler material might be a standard density silicone gel or a reduced density silicone gel with microspheres added. Regardless or the material selected, the filler material is poured into the mold and allowed to sit for an appropriate duration to allow any undesired voids or air bubbles to dissipate. At this point, the mold is again placed into the heat chamber to cure, preferable at the same temperature as that of the elastomer. An example for the external breast prosthesis would be at 85° C. for an additional ten minutes.

After the resin-based filler material has cured, the mold is again removed from the oven. At this point, another coating of the resin-based elastomer is sprayed onto the back surface of the article (corresponding with the open face of the mold) to create the outer ‘skin’ or film on the rear side of the product. For instance, on the surface of the external breast prosthesis that would fit against the wearer's body. Again, the elastomer is sprayed by an appropriate method and for an appropriate duration to give the desired thickness of the ‘skin’ of the article. At this point, the mold is place back into the heat chamber for the final cure of the most recently applied elastomer. For example, the mold for an external breast prosthesis may be placed into the heat chamber for an additional five minutes at a temperature of 85° C.

Finally, after the article is completely cured, the mold is removed from the heat chamber and allowed to cool. The external breast prosthesis, or whatever the formed resin-based article may be, is then removed from the mold, and any excess elastomer sheathing is trimmed from the article.

In the external breast prosthesis embodiment, this process eliminates the time and expense of forming and using thermoplastic films to sheathe the gelatinous prosthesis, while retaining the benefits of a prosthesis sheathed in film. Those benefits being a fully crosslinked article that is not susceptible to seepage of the resin based material.

Although the specific example disclosed throughout the specification mainly deals with external breast prostheses, and number of other prostheses, cushions, and pads are contemplated to fall within embodiments of the present invention, including, but not limited to facial prostheses, other soft tissue prostheses, wheel-chair cushions, ergonomic wrist pads, and other pads and cushions for various medical devices.

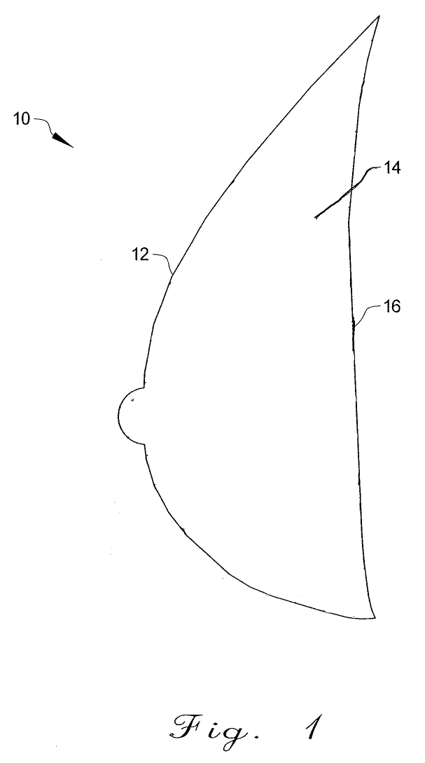

An article so derived from the process of the present invention is shown in FIG. 1 and generally denoted by the numeral 10. Referring to FIG. 1, a first resin-based sheathing (12) is shown, cured and chemically bonded to cured, resin-based filler material (14). Finally, a second resin-based sheathing (16) is shown, cured and chemically bonded to cured, resin-based filler material (14) as well. Additionally, as can be seen in FIG. 1, first resin-based sheathing (12) is bonded to second resin-based filler material (16) about the perimeter of article (10).

Although the invention has been described with reference to specific embodiments, this description is not meant to be construed in a limited sense. Various modifications of the disclosed embodiments, as well as alternative embodiments of the inventions will become apparent to persons skilled in the art upon reference to the description of the invention. It is, therefore, contemplated that the appended claims will cover such modifications that fall within the scope of the invention.

I claim:

1. A method of forming a resin-based article, comprising the steps of:
selecting a mold, said mold having a concave surface with an open face;
selecting a resin-based elastomer;
applying a first coating of said resin-based elastomer to said concave surface of said mold;
curing said first coating of resin-based elastomer;
selecting resin-based filler material;
adding said filler material to said mold;
curing said filler material in said mold;
applying a second coating of said resin to said filler material; and
curing said second coating of resin-based elastomer.

2. The method of claim 1 wherein said resin-based elastomer is capable of creating a bond with said resin-based filler material.

3. The method of claim 2 wherein said resin-based elastomer is a silicone based elastomer diluted into a liquid form with silicone crosslinker added and said resin-based filler material is a silicone gel based material.

4. The method of claim 2 wherein said resin-based elastomer is a polyurethane based elastomer diluted into a liquid form and said resin-based filler material is a polyurethane based foam material.

5. The method of claim 2 wherein said first coating of said resin-based elastomer is applied to said concave surface of said mold via a painting operation.

6. The method of claim 2 wherein said first coating of said resin-based elastomer is applied to said concave surface of said mold via a spraying operation.

7. The method of claim 6 wherein said first coating of said resin-based elastomer is applied to said concave surface of said mold in a uniform thickness.

8. The method of claim 6 wherein said first coating of said resin-based elastomer is applied to said concave surface of said mold in a non-uniform thickness.

9. The method of claim 2 wherein said first and second coatings of said resin-based elastomer are thermally cured within a heat chamber.

10. The method of claim 9 wherein said resin-based filler material is thermally cured within a heat chamber.

11. A resin-based article, comprising:
a first resin-base elastomer sheathing;
a filler material, said filler material being juxtaposed to and bonded with said first resin-based elastomer sheathing; and
a second resin-based elastomer sheathing, said second resin-based elastomer sheathing being juxtaposed to and bonded with said filler material, said second resin-based elastomer being bonded with said first resin-based elastomer sheathing about the perimeter of said first and second resin-based elastomer sheathings.

12. The article of claim 11 wherein said article is formed by the steps of:
selecting a mold, said mold having a concave surface with an open face;
selecting a resin-based elastomer;
applying a first coating of said resin-based elastomer to said concave surface of said mold;
curing said first coating of resin-based elastomer;
selecting resin-based filler material;
adding said filler material to said mold;
curing said filler material in said mold;
applying a second coating of said resin to said filler material; and
curing said second coating of resin-based elastomer.

13. The article of claim 12 wherein said resin-based elastomer is capable of creating a bond with said resin-based filler material.

14. The article of claim 13 wherein said resin-based elastomer is a silicone based elastomer diluted into a liquid form with silicone crosslinker added and said resin-based filler material is a silicone gel based material.

15. The article of claim 13 wherein said resin-based elastomer is a polyurethane based elastomer diluted into a liquid form and said resin-based filler material is a polyurethane based foam material.

16. The article of claim 13 wherein said first coating of said resin-based elastomer is applied to said concave surface of said mold via a painting operation.

17. The article of claim 13 wherein said first coating of said resin-based elastomer is applied to said concave surface of said mold via a spraying operation.

18. The method of claim 17 wherein said first coating of said resin-based elastomer is applied to said concave surface of said mold in a uniform thickness.

19. The method of claim 17 wherein said first coating of said resin-based elastomer is applied to said concave surface of said mold in a non-uniform thickness.

20. The method of claim 13 wherein said first and second coatings of said resin-based elastomer are thermally cured within a heat chamber.

21. The method of claim 20 wherein said resin-based filler material is thermally cured within a heat chamber.
